(19) United States
(12) Patent Application Publication
(10) Pub. No.: US 2013/0225950 A1
(43) Pub. Date: Aug. 29, 2013

(54) ANXIETY MONITORING

(71) Applicants: Gijs Antonius Franciscus Van Elswijk, Eindhoven (NL); Nathalie Magali Danielle Dessaud, Eindhoven (NL); Tim Johannes Willem Tijs, Helmond (NL); Murray Fulton Gillies, Eindhoven (NL); Jürgen Vogt, Eindhoven (NL)

(72) Inventors: Gijs Antonius Franciscus Van Elswijk, Eindhoven (NL); Nathalie Magali Danielle Dessaud, Eindhoven (NL); Tim Johannes Willem Tijs, Helmond (NL); Murray Fulton Gillies, Eindhoven (NL); Jürgen Vogt, Eindhoven (NL)

(73) Assignee: KONINKLIJKE PHILIPS ELECTRONICS N.V., EINDHOVEN (NL)

(21) Appl. No.: 13/879,836

(22) PCT Filed: Oct. 12, 2011
(86) PCT No.: PCT/IB2011/054496

§ 371 Date: Apr. 17, 2013

(30) Foreign Application Priority Data
Oct. 19, 2010 (EP) 10188038.3

(51) Int. Cl. A61B 5/145

(52) U.S. Cl. CPC A61B 5/14546; A61B 5/14507
USPC 600/309

(57) ABSTRACT

A patient monitoring system comprises a probe module (10) to acquire data of a patient to be monitored. An evaluation module (20) to determines a patient's state of anxiety on the basis of the acquired data. An indicator module (30) to feedback the determined state of anxiety to attending staff. Thus, the staff is enabled on the basis of the feedback from the patient monitoring system to re-assure patients where that is needed and beneficiary.

### FIELD OF THE INVENTION

The invention pertains to a patient monitoring system having the function to assess stress or anxiety with a patient to be examined.

### BACKGROUND OF THE INVENTION

Such a patient monitoring system is known from the paper ‘Clinical decision—support for diagnosis stress-related disorders by applying psychophsiological medical knowledge to an instance—based learning system’ by M. Nilsson et al. in Artificial Intelligence in Medicine 36 (2006) 159-176. This known patient monitoring system classifies heart-rate patterns. The classified heart-rate patterns indicate if a patient may be suffering from a stress-related disorder.

### SUMMARY OF THE INVENTION

An object of the present invention is to efficiently and effectively put a patient to be examined at ease. Notably, the present invention addresses to reduce patient's level of anxiety when the patient needs to undergo an examination in an imaging modality such as an x-ray examination system, a magnetic resonance examination system, a PET system, a computed tomography system or a hybrid imaging system such as a PET/MRI system or a PET/CT system.

This object is achieved by a patient monitoring system of the invention comprising
a probe module (10) to acquire data of a patient to be monitored
an evaluation module (20) to determine a patient's state of anxiety on the basis of the acquired data and
an indicator module (30) to feedback the determined state of anxiety to attending staff.

On the basis of the determined state of patient anxiety, the attending staff is informed. Notably, the state of anxiety includes the level of anxiety of the patient in a quantitative measure. Consequently, the staff can efficiently and effectively approach the more anxious patients in a re-assuring way to put those patients at ease. Thus, the staff is enabled on the basis of the feedback from the patient monitoring system to re-assure patients where that is needed and beneficiary. Notably, the invention avoids that staff approaches patients that are not so anxious and incur risks that those patients are rendered more anxious because of unnecessary staff attention . Further the invention reduces exposure of hospital staff to radiation and thus avoids that hospital staff is unnecessarily exposed to radiation (in for example nuclear medicine (PET) examinations).

The invention enables a more reliable assessment of the patient's anxiety level because the assessment is not affected by personal interaction between member(s) of staff and the patient. The staff's attention is accurately customised to the individual patient because the feedback to the staff is based on the data acquired from the patient at issue. In this way, extra time spent by staff on an objectively more anxious patient will lead to a benefit in less time required for the patient who is more susceptible to anxiety during the actual medical procedure.

Examples of such medical procedure are imaging examinations e.g. by magnetic resonance imaging or positron emission tomography that requires a radioactive tracer. These are expensive imaging procedures for which it is to be avoided that they would need to be re-iterated because of restlessness of the anxious patient. Another benefit of the patient monitoring system of the invention is that it can help minimize exposure time of hospital staff to radiation in medical procedures that involve radioactive tracers. Finally, this invention provides a more personalized patient care which contributes to overall improvement of patient well-being.

The invention takes account of a wide variety of anxiety levels of different patients and different types of medical procedures. In particular, individual patients may react differently on various types of medical procedures, like an imaging examination procedure based on x-ray imaging, computed tomography or magnetic resonance imaging.

Further, the invention facilitates formal implementation of an anxiety management protocol in a healthcare institution, such as in a hospital or point of care, because it allows different anxiety management strategies to different anxiety profiles, coping styles, and anxiety levels. The invention enables to generate decisions to be better customised to the patient's needs and to give a patient extra attention independent form a individual subjective anxiety assessment of the physician or nurse involved at any particular time. Thus the way a patient is approached or guided can vary extensively.

The recommendations or assessments of this invention create more consistency in patient-staff contact in a given department. Once a patient profile has been determined it can be stored to help improve efficiency in planning of return visits, for example by avoiding planning of anxious patients at a concurrent timeslot. The feedback to the staff may be provided in different forms. For example the patient monitoring system includes a user interface with a display on which the patient's level of anxiety can be displayed. An attractive way of displaying the patient's level of anxiety is as a ‘traffic light’. For procedures where multiple patients are monitored or the nurse is mobile then a portable display device (e.g. “iPhone”or “iPad”) can be the preferred interface.

These and other aspects of the invention will be further elaborated with reference to the embodiments defined in the dependent Claims.

In one aspect of the patient monitoring system of the invention, the probe module is configured to acquire primary data which are employed by the evaluation module to generate a primary assessment of the patient's level of anxiety. Preferably, also a reliability score for the assessed level of anxiety is calculated. The reliability score can be a statistical spread measure such as the standard deviation, or coefficient of variation.

Instead of the above simple statistical spread measure, more sophisticated algorithms might be used to combine information from multiple sources to determine the probability and/or reliability scores, such as conditional or Bayesian probabilities, decision trees, etc.

When on the basis of the primary assessment of the patient's anxiety level, optionally also taking the calculated confidence range into account, additional data may be acquired and employed to make a more refined assessment of the patient's anxiety level. In particular, in this aspect of the invention, the primary data acquisition is less obtrusive for the patient and/or the staff. Thus, it is achieved that more obtrusive additional data acquisition is activated only when there is a real need to do so. In other words, more obtrusive data acquisition is avoided when the patient's anxiety level could be reliably assessed on the basis of the primary data, which only requires a less obtrusive approach of the patient.

In a further aspect of the invention the patient monitoring system of the invention is provided with a self-learning function. In this aspect the patient monitoring system is provided with a user interface via which the staff can confirm or override the evaluated anxiety level. This input from the staff is stored and used to update the relationship between the acquired data and the to be evaluated anxiety level. Then the updated relationship is stored and made available to the evaluation module for future use. This self-learning aspect may be assigned to an individual patient but can also be used in the evaluation of anxiety of any patient from a wider group. For example, over time it might become evident that anxiety levels for lymphoma patients on their first PET-scan is typically much higher than in follow-up scans. In another aspect of the invention, the evaluation module is provided with, or provided with an access to, stored patient reference data that are related to pre-determined anxiety levels. Multiple parameters in the reference dataset that are related to the pre-determined anxiety level yield more reliable estimates of the anxiety level of the patient. Examples of reference data are demographic profiles that include patient's gender, age, smoking behaviour, education level, or personality traits such as trait-anxiety or preferred coping style.

In another aspect of the invention the probe module is provided with a camera system that acquires image information of the patient. Notably, the camera system can be configured to image several patients simultaneously, e.g. while they are in the waiting room before undergoing the medical procedure. From the image information several parameters of the imaged patient can be derived, such as heart rate, respiration rate, motion, facial expression, pupil size (diameter). Image based respiration monitoring as such is mentioned in the European patent application 10160571.5 (PH014905). By comparing one or more of these parameters to reference distributions the anxiety level of the patient can be assessed

In a further aspect of the invention, the probe module is provided with a physiology sensor. The evaluation module evaluates the patient's level of anxiety from the physiological data acquired by the physiology sensor. For example, the physiology sensor may be a temperature sensor to measure the patient's skin temperature, or a conductivity sensor (Ohm meter) to measure the electrical conductivity of the patient's skin.

In a next aspect of the invention, the probe module is configured to present the patient with a questionnaire. Example of a typical questionnaire is the Spielberger's State Anxiety Inventory that consist of 20 questions, such as: “I feel calm”, “I feel secure”, “I am tense”. Each of the questions has to be responded to on a discrete 5-point scale (called a Likert scale) from 1 (not at all) to 5 (very much so). The evaluation module derives the level of anxiety from the responses to the questionnaire. For most psychological questionnaires norm tables are published for several reference populations (e.g. males, elderly, students, etc).

In a specific aspect of the invention, the probe module includes a cortisol assay which measures the cortisol in the patient's saliva. Alternatively, blood cortisol level may be employed. It is known per se that psychological stress, leads to the release of corticotropin releasing hormone and adrenocorticotropic hormone with a subsequent rise in cortisol levels Kirschbaum, C., & Hellhammer, D. (1994). ‘Salivary cortisol in psychoneuroendocrine research:Recent developments and applications.’ in Psychoneuroendocrinology, 19 (1994) 313-333.

In a further aspect of the invention, the evaluation module is further configured to take availability of staff into account for providing feedback on the patient's state of anxiety to the staff. In this implementation the evaluation module has access to e.g. staff work schedule as planned or information of staff's current activities. For example the evaluation module is configured to lower feedback if there is less staff available and provide feedback limited to patient's showing higher levels of anxiety. In this way the patient monitoring system of the invention improves the efficiency of the deployment of staff Notably, when staff is more busy, they are less provided with feedback on lesser anxious patient's because there will not be staff available to attend and re-assure those lesser anxious patients. On the other hand, the more staff is available, the evaluation module controls the indicator module to also provide feedback on the lesser anxious patients.

In another aspect, the invention is applied in the field of nuclear medicine, in particular in conjunction with PET (positron emission tomography. In this embodiment the probe module has the further function to measure radioactivity of a radioactive tracer contrast agent prepared to be administered to the patient to be examined. The radioactivity can be measured with a semiconductor radiation sensor or with a Geiger counter type of device. The measured level of radioactivity is applied to the evaluation module. The evaluation module is configured to determine an optimum instant for administering, e.g. by injection or infusion, the radioactive tracer contrast agent and the patient's state of anxiety. An object of application of the invention in nuclear medicine is to improve clinical workflow. It is observed that in nuclear medicine conventional practices to reduce stress, such as massage therapy, are not conventionally applied because of the risk to deteriorate the uptake of radioactive contrast agent. In nuclear medicine, the workflow is dominated by the requirements due to the half-life of the radioactive tracer contrast agent. For example radioactive ¹⁸F has a rather short half-life of about 110 minutes and needs to be administered to the patient e.g. in the form of fluor-deoxyglucose (¹⁸FDG) rather short before the actual PET scan is performed. The present invention enables to apply the radioactive contrast agent optimally when the patient is relatively relaxed while the radioactivity is relatively high. Because patient anxiety (or stress) level is lower, perturbations due to patient stress on the acquired image data are avoided and the diagnostic image quality is improved. In particular, as the patient's anxiety level is lower, tumour-background radioactivity in the image data is higher and the level of false positives in the image is lower. Notably, as the anxiety level of the patient to be examined is lower, abnormal hypermetabolistic areas that may not contain cancer cells, but are e.g. due to muscular activity is avoided. On the other hand, when the instant for administering the radioactive contrast agent is optimised, excessive exposure to radioactivity can be avoided without negatively affecting diagnostic image quality. Accordingly, the invention enables to optimise the instant of administering the radioactive contrast agent, reduce the anxiety level of the patient during uptake of the contrast agent as well as during the actual acquisition of image data, e.g. during the PET scan. These effects are moreover achieved together with in improved clinical workflow in which attending staff is more efficiently deployed and less exposed to radioactivity. Workflow is even further improved by taking staff availability into account together with the measured radioactivity. In a practical example of the invention, the probe module is formed as a bracelet to be worn by the patient. The bracelet or wristband incorporates both a sensor to acquire the data of the patient from which the state of anxiety is determined as well as a radioactivity sensor. For example, a semiconductor radiation sensor and a senor for measuring electrical skin conductivity can be incorporated in such a bracelet. The bracelet is non-obtrusive for the patient and does not inherently add to the patient's anxiety.

These and other aspects of the invention will be elucidated with reference to the embodiments described hereinafter and with reference to the accompanying drawing wherein

### BRIEF DESCRIPTION OF THE DRAWINGS

FIG. 1 shows a diagrammatic representation of a patient monitoring as implemented at a point-of-care and

FIG. 2 another example of invention applied in nuclear medicine is schematically depicted.

### DETAILED DESCRIPTION OF THE EMBODIMENTS

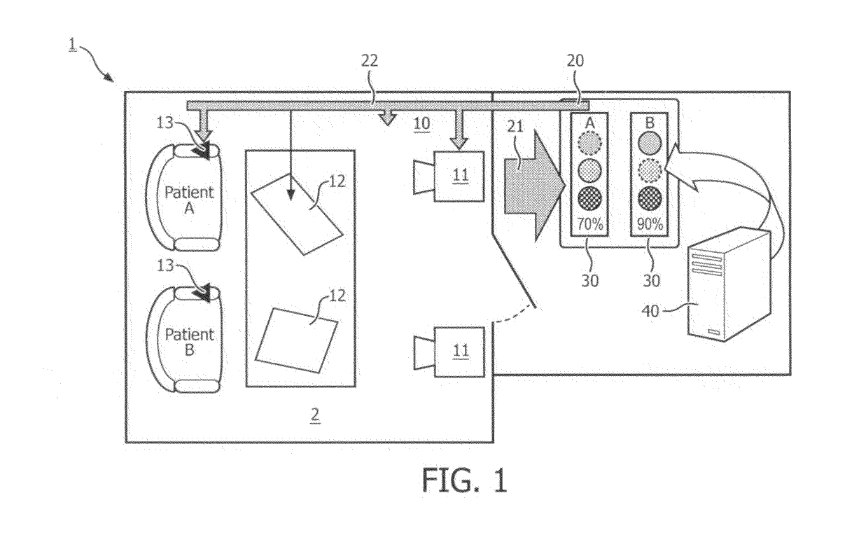

FIG. 1 shows a diagrammatic representation of a patient monitoring as implemented at a point-of-care, such as a physician's office or an imaging modality suite. The patient monitoring system 1 of the invention comprises the probe module 10, which acquires data from the patient to be examined. In the example of FIG. 1 two camera's 11 are provided to image the motion due to heart rate, respiratory pattern, motion, pupil size (diameter) and the facial expression of the patient. From an image analysis of these images, the anxiety level of the patient is assessed. To that end the evaluation module is provided with an image analysis and an assessment function that is usually implemented in software. In the set up shown in FIG. 1, two patients, patient A and patient B, in the waiting room 2, can be monitored individually. For each of the patient's a photoplethysmography sensor 13 is provided to acquire the patient's pulse rate. Further, questionnaire consoles 12 are provided which dispense access to questionnaires for each of the patients. For example state anxiety questionnaires or trait anxiety questionnaire may be employed. The questionnaire consoles output the patient's responses to these questionnaires. In secondary stages a “trait” questionnaire is used that will provide information about relevant personality characteristics such as coping style, or trait anxiety. Examples would be: Hospital Anxiety and Depression Scale; Spielberger's Trait Anxiety Inventory and Coping Operations Preference Enquiry.

The data acquired by the probe module 10 are applied to the evaluation module 20 over a data connection 21. On the basis of the data from the probe module, the evaluation module computes anxiety estimates for the individual patients. The evaluation module supplies its output anxiety estimate to the indicator module 30, for example in the form a ‘traffic lights’ for each individual patient. Thus the evaluation module assesses the patient's anxiety level and feedbacks the assessment to the staff. The ‘traffic lights’ may be further provided with a feedback option, e.g. in the form of a button that can be applied by the staff in response to the indication generated by the evaluation module 20. The staff's feedback may be to confirm or to overrule the evaluation module's assessment. The staff's feedback is used by the evaluation module's self-learning function to improve its accuracy.

Further, the evaluation module 20 is provided with access to stored reference data in a memory unit 40. The memory unit contains demographic profiles, including the patient's gender, age, type of examination, total number of visits.

The evaluation module also has the function to assess the patient's anxiety level at several hierarchical levels and control the probe module to perform additional data in conformity with the hierarchy level of the assessment. The hierarchical levels involve for example a primary, and further more increasingly refined assessments that require primary and additional data, respectively, to the acquired. For example, already in the primary assessment a reliability score of the assessed anxiety level is calculated on the basis of which the next hierarchical level is activated. In an example of this: Patient is an adult male. The first variable is the heart rate measured at 120 beats/minute. The evaluation system knows the probability distribution function of the heart rate for stressed males, and thereby determines that the probability that a male under stress has a heart rate ≧120 is 90%. In a similar fashion, the stress probability is computed for a second and third variable (e.g. respiration rate and pupil size). For these variables the respective stress probabilities are 40% and 80%. This leads to the average stress probability of 70% (i.e. a red traffic light), with the standard deviation of 26% as a reliability index.

Increasing hierarchy has an increasing level of obtrusiveness for the patient to be examined. To control raising to the next level, the patient monitoring system comprises a control bus over which control signals are transferred from the evaluation module 20 to the probe module 10, notably to the data acquisition components of the probe module, such as the camera's, questionnaire consoles etc. The evaluation module is configured to turn to a higher hierarchical level if the assessment at the current level has a confidence level below pre-set threshold.

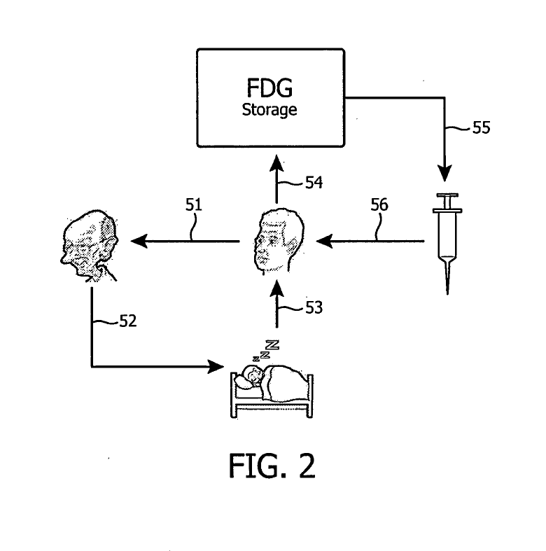

Another example of invention applied in nuclear medicine is schematically depicted in FIG. 2: Patient stress level is communicated at an early stage to the clinician 51, to help the clinician take the appropriate steps (e.g., schedule extra time if expected necessary) for patient relaxation. Next, the clinician aims to relax the patient, possibly assisted by technology 52>53. As soon as the injection system notices the patient is sufficiently relaxed, the FDG storage unit is triggered 54. The FDG storage unit determines the optimal FDG quantity (given the desired dose and current radioactivity) and sends the

FDG to the injection unit 55, which injects the FDG into the patient 56.

1. A patient monitoring system comprising
a probe module to acquire data of a patient to be monitored, wherein the probe module includes a salivary cortisol assay to measure a salivary cortisol level or blood cortisol level,
an evaluation module to determine a patient's state of anxiety on the basis of the acquired data, the evaluation module configured to determine the patient's state on the basis of the salivary cortisol level or blood cortisol level, and
an indicator module to feedback the determined state of anxiety to attending staff.

2. The patient monitoring system as claimed in claim 1, wherein
the probe module is configured to acquire primary data and optionally to acquire additional data of the patient
the evaluation module is configured
to make a primary assessment of the patient's state of anxiety from the primary data and optionally make a refined assessment of the patient's state of anxiety on the basis of the additional data and
to control the probe module on the basis of the primary assessment to acquire the additional data and to engage in the refined assessment.

3. The patient monitoring system as claimed in claim 1, wherein the evaluation module includes a self-learning function.

4. The patient monitoring system as claimed in claim 1, wherein the evaluation module is configured to access patient reference data and to evaluate the patient's state taking the patient reference data into account.

5. The patient monitoring system as claimed in claim 1, wherein the probe module includes a camera to monitor the patient and an image-evaluation module to evaluate the patient's state of anxiety on the basis of image information acquired by the camera.

6. The patient monitoring system as claimed in claim 1, wherein the probe module includes a physiology sensor and an physiology-evaluation module to evaluate the patient state of anxiety on the basis of physiological data acquired by the physiology sensor.

7. The patient monitoring system as claimed in claim 1, wherein the probe module is arranged to present a questionnaire to the patient and receive responses to the questionnaire.

8. (canceled)

9. The patient monitoring system as claimed in claim 1, wherein the evaluation module is further configured to take staff availability into account for controlling the indicator module to feedback the state of anxiety.

10. The patient monitoring system as claimed in claim 1, wherein
the probe module also has the function to measure radioactivity of a radioactive tracer prepared to be administered to the patient and
the evaluation module is configured to
receive the measured radioactivity level ,
to determine an optimum instant for administering the radioactive tracer to the patient on the basis of the measured radioactivity level and the state of anxiety of the patient.

11. The patient monitoring system as claimed in claim 1, wherein
the probe module has the function to measure radioactivity of a radioactive tracer that has been administered to the patient and
the evaluation unit is configured to control the indicator module to feedback to the attending staff on the basis of the patient's state of anxiety and the measured level of radioactivity.

12. A patient monitoring method including
acquisition of data of a patient to be monitored including a cortisol assay which measures the cortisol in the patient's saliva or blood
determination of an indication of a patient's state of anxiety on the basis of the acquired data, and
an indicator module to feedback the determined indication of the state of anxiety to attending staff.

13. A computer programme including instructions to
acquire data of a patient to be monitored, including the cortisol in the patient's saliva or blood,
determine a patient's state of anxiety and feedback the determined state of anxiety to attending staff.
